(19) United States
(12) Patent Application Publication
(10) Pub. No.: US 2002/0037877 A1
(43) Pub. Date: Mar. 28, 2002

(54) Pharmaceutical suspension compositions lacking a polymeric suspending agent

(75) Inventor: Onkar N. Singh, Arlington, TX (US)

Correspondence Address: Alcon Universal Ltd., c/o Alcon Research, Ltd., Patrick M. Ryan(Q-148), R&D Counsel, 6201 So. Freeway, Fort Worth, TX, 76134-2099, US

(73) Assignee: Alcon Universal Ltd.

(21) Appl. No.: 09/906,219

(22) Filed: Jul. 16, 2001

Related U.S. Application Data
(60) Provisional application No. 60/220,753, filed on Jul. 26, 2000.

(51) Int. Cl. A61K 31/685; A61K 31/573; A61K 31/55

(52) U.S. Cl. 514/78; 514/179; 514/217; 514/256

(57) ABSTRACT

Stable aqueous pharmaceutical suspension compositions containing lecithin as a stabilizing additive and lacking a polymeric suspending agent are disclosed.

This application claims priority to U.S. Provisional Application Serial No. 60/220,753, filed Jul. 26, 2000.

### BACKGROUND OF THE INVENTION

The present invention relates to pharmaceutical suspension compositions. In particular, this invention relates to physically stable aqueous pharmaceutical compositions of water-insoluble drugs.

### DESCRIPTION OF RELATED ART

Aqueous pharmaceutical suspension compositions typically contain one or more polymeric suspending or viscosity-enhancing agents to enhance physical stability. The polymeric suspending agents, which can be ionic or nonionic, help keep the water-insoluble components of the composition suspended. The polymeric suspending agents also make it easier to resuspend the composition after water-insoluble components have settled to the bottom of a container.

Many polymeric suspending agents are known. Polymeric suspending agents commonly used in aqueous pharmaceutical suspension compositions include carbomers, polyvinyl alcohol, polyvinyl pyrrolidone, hydroxypropylmethyl cellulose, hydroxyethyl cellulose, hydroxypropyl cellulose, methyl cellulose, carboxymethyl cellulose, microcrystalline cellulose, powdered cellulose, xanthan gum, gellan gum, carageenan, acacia, tragacanth, gelatin, guar gum, alginic acid, sodium alginates, propylene glycol alginate, eudragit (methacrylic acid and methyl methacrylate copolymer), dextrin, dextran, dextran-polyethylene glycol conjugates, and the glycosaminoglycans family of polymers, such as heparin sulfate, heparan sulfate, dermatan sulfate, chondroitin sulfate.

U.S. Pat. No. 5,843,930 discloses topically administrable ophthalmic and otic compositions comprising (a) ciprofloxacin in aqueous solution in an amount effective for antibacterial action; (b) a non-ionic viscosity augmenter unaffected by pH and ionic level, said viscosity augmenter being present in an amount effective for augmenting the viscosity of the composition to a viscosity greater than that of water, said viscosity augmenter being at least 85% hydrolyzed polyvinyl alcohol; (c) a non-ototoxic preservative present in an amount effective for antibacterial action the preservative being benzyl alcohol; (d) water sufficient to produce an aqueous composition; (e) hydrocortisone in aqueous suspension in an amount effective for anti-inflammatory action; (f) lecithin in an amount effective for enhancing suspension of other constituents in the compositions; and (g) polysorbate ranging from polysorbate 20 to 80 in an amount effective for spreading the preparation on a hydrophobic skin surface to the site of infection or inflammation.

According to the '930 patent, the compositions comprising ciprofloxacin and hydrocortisone contain polyvinyl alcohol in an amount effective for augmenting the viscosity of the composition to a viscosity greater than that of water and suspending other constituents of the composition. To allow a ciprofloxacin preparation to be administered in drops from a medicine dropper and to flow by gravity to and remain or deposit in an effective amount at a selected area, a viscosity-augmenting agent that would also serve to suspend hydrocortisone was desirable. For compatibility with ciprofloxacin hydrochloride solubility, viscosity-augmenting agents were preferably non-ionic and unaffected by pH and ionic level. See Col., 8, lines 13-31 of the '930 patent.

Polyvinyl alcohol was selected for its ability to produce a suitable viscosity and a high ability to suspend hydrocortisone in aqueous preparations. See the '930 patent at Col. 8, lines 32-37. The addition of lecithin to the composition enhanced the efficacy of polyvinyl alcohol in suspending hydrocortisone in aqueous preparations with ciprofloxacin hydrochloride and other components. See the '930 patent at Col. 8, line 64-Col. 9, line 12.

The '930 patent discloses a process for manufacturing compositions containing ciprofloxacin and hydrocortisone in Example 5 at Column 5, lines 27-67. According this manufacturing process, polyvinyl alcohol, lecithin, benzyl alcohol and acetic acid are sequentially added to prepare a first stock solution. Separately sodium chloride and sodium acetate are dissolved in water to form a second stock solution. A third stock solution is prepared by dissolving polysorbate 20 and dispersing hydrocortisone in water. Finally, ciprofloxacin is either added to the first stock solution or ciprofloxacin is prepared as a fourth stock solution by dissolving ciprofloxacin, acetic acid and sodium acetate to form a ciprofloxacin stock solution. After the first and second stock solutions are combined, the ciprofloxacin stock solution is added to the combined solution. Finally, the third stock solution polysorbate 20 and hydrocortisone is mixed with the remaining batch volume.

A suspension composition's physical stability can be measured by two common methods. First, the resuspendability of a composition can be measured by allowing a homogeneous to remain standing in a cylindrical container for a period of time, then determining the number of inversions of the cylindrical container necessary to resuspend any sediment that form while the composition was standing. Second, the rate of settling can be measured by allowing a homogeneous suspension composition to remain standing for a period of time, then observing the height of sedimentation visible in a sample contained in a cylinder. Larger sedimentation heights indicate less separation with less supernatant liquid. Both measures of physical stability are important. A composition that is very easy to redisperse but that settles too quickly can be difficult to manufacture. Suspension compositions must remain well dispersed during processing and filling operations while commercial supplies are prepared in order to insure uniform products.

### SUMMARY OF THE INVENTION

The present invention provides aqueous pharmaceutical suspension compositions that have excellent physical stability. The compositions contain one or more drugs that are insoluble or sparingly soluble in water such that at least a portion of the drug compound(s) contained in the compositions of the present invention is intended to be suspended. The compositions contain a physical-stability enhancing additive consisting essentially of lecithin.

The present invention also relates to a method of preparing an aqueous pharmaceutical suspension composition comprising lecithin but lacking a polymeric suspending agent. According to the present invention, a water-insoluble drug compound is mixed in a lecithin dispersion prior to being combined with the balance of the aqueous suspension composition.

Among other factors, the present invention is based upon the finding that a specific order of addition of ingredients in compositions containing a water-insoluble drug and lecithin but lacking a polymeric suspending agent provides such compositions with excellent physical stability. Compositions prepared by dispersing a water-insoluble drug with lecithin prior to mixing the drug with the balance of ingredients in the compositions have superior physical stability compared to those prepared by combining all ingredients in one step or by dispersing the water-insoluble drug with only a surfactant prior to mixing the drug with the balance of the composition.

### DETAILED DESCRIPTION OF THE INVENTION

Unless otherwise indicated, all ingredient concentrations are listed as percent (w/w).

As used herein, “water-insoluble drug compound” means a drug compound that is insoluble or poorly soluble in water such that in the final pharmaceutical composition at least a portion of the total amount of the drug compound is intended to be in suspension rather than in solution.

As used herein, “physical-stability enhancing additive consisting essentially of lecithin” means that the suspension composition contains lecithin but lacks a polymeric suspending agent or polymeric viscosity-enhancing agent. Typical polymeric suspending agents or polymeric viscosity-enhancing agents include carbomers, polyvinyl alcohol, polyvinyl pyrrolidone, hydroxypropylmethyl cellulose, hydroxyethyl cellulose, hydroxypropyl cellulose, methyl cellulose, carboxymethyl cellulose, microcrystalline cellulose, powdered cellulose, xanthan gum, gellan gum, carageenan, acacia, tragacanth, gelatin, guar gum, alginic acid, sodium alginates, propylene glycol alginate, eudragit (methacrylic acid and methyl methacrylate copolymer), dextrin, dextran, dextran-polyethylene glycol conjugates, and the glycosaminoglycans family of polymers, such as heparin sulfate, heparan sulfate, dermatan sulfate, chondroitin sulfate.

The compositions of the present invention contain a therapeutic or prophylactic amount of one or more water-insoluble drug compounds. The amount of such water-insoluble drug compounds depends on a number of factors including individual drug potency, targeted indication, etc. Typical drug concentrations range from about 0.001-5%. Many water-insoluble drugs are known, including steroids such as dexamethasone; rimexolone; prednisolone; hydrocortisone; fluticasone propionate; budesonide; mometasone furoate monohydrate; and dexamethasone beloxil. Water-insoluble compounds other than steroids include griseofulvin; carbamazepin; clofibrate; ketoprofen; 5-flurouracil; flurbiprofen; mefanamic acid; flufenamic acid; and crystalline beta escinic acid.

Particularly for topical ophthalmic use, small particle sizes of the water-insoluble drug are preferred. As used herein, “micronized” drug particles means drug particles having an average particle size ≦10 μm (based on surface area (dsn)). If the particle size of the drug raw material as received from the supplier is unsatisfactory, one or more known sizing techniques, such as ball milling or micronizing, can be used to adjust the particle size into the desired range.

To enhance the physical stability of the suspension composition of present invention, the composition contains a physical-stability enhancing additive consisting essentially of lecithin or a lecithin derivative. Lecithins from natural/vegetative (e.g., egg or soy lecithin) and synthetic origins are known. The primarily type of lecithin is phosphatidylcholine (PC). Other types of lecithins include phosphatidylglycerol; phosphatidylinositol; sphingomyelin; and phosphatidylethanolamine. Derivatives of lecithin with saturated and unsaturated fatty acid side chains on PC, are also known, including: distearoylphosphatidyl choline; dipalmitoylphosphatidyl choline; and dimirystoylphosphatidyl choline. As used herein, “lecithin” includes such derivatives of lecithin. Preferably, the lecithin ingredient comprises at least 75% PC.

Commercially available grades of soy lecithins include a fully hydrogenated soy lecithin comprising 90% phosphatidylcholine available under the tradename Phospholipon 90H from American Lecithin Company and a soy lecithin comprising 75% phosphatidylcholine available under the tradename Lipoid-S75 from Vernon Walden, Inc. The amount of lecithin contained in the compositions of the present invention depends primarily on the concentration of insoluble ingredients in the compositions. The amount of lecithin in the compositions of the present invention generally ranges from about 0.01-5%, preferably about 0.01-2% and most preferably is about 0.15%.

In addition to the water-insoluble drug compound and lecithin, the compositions of the invention preferably contain a non-ionic surfactant. The most preferred nonionic surfactants are the surfactants known as polysorbates, in particular polysorbates 20-80. Such polysorbate surfactants are commercially available under the tradename Tween from ICI Americas, Inc. Most preferred is polysorbate 20. The amount of surfactant contained in the compositions of the present invention generally ranges from about 0.01-2%, preferably about 0.05-1%, and most preferably is about 0.1%.

In addition to the water-insoluble drug compound, lecithin and optional surfactant, the compositions, if intended for topical ophthalmic use, contain a tonicity-adjusting agent. The tonicity-adjusting agent is present in an amount sufficient to cause the final composition to have an ophthalmically acceptable osmolality (generally about 150-450 mOsm, preferably 250-350 mOsm). If desired or required, the compositions of the present invention also contain one or more excipients. Conventional excipients include preservatives, buffering agents, chelating agents or stabilizers, viscosity-enhancing agents and others. The chosen ingredients are mixed until homogeneous. After the solution is mixed, pH is adjusted (typically with NaOH or HCl) to be within a range suitable for the intended pharmaceutical use, generally within the range of pH 4.5-8.

Sodium chloride, mannitol, glycerin or the like may be used as the isotonic agent; benzalkonium chloride, polyquaternium-1, benzyl alcohol or the like as the preservative; sodium hydrogenphosphate, sodium dihydrogenphosphate, boric acid or the like as the buffering agent; edetate disodium or the like as the chelating agent or chemical stabilizer; and sodium hydroxide, hydrochloric acid or the like as the pH controller.

The compositions of the present invention are preferably applied topically to the eye, ear or nose, but could be used elsewhere for topical or injected application.

The compositions of the present invention are prepared in a specific manner. It is essential that the water-insoluble drug compound is first mixed with lecithin prior to being combined with the remainder of the composition. Preferably, the water-insoluble drug compound is mixed with both lecithin and a nonionic surfactant (preferably polysorbate 20 to 80) before being combined with the remainder of the composition. The presence of the surfactant provides a lower viscosity slurry than simply mixing hydrocortisone and lecithin alone. The lower viscosity achieved by the addition of the surfactant makes processing easier.

If not available as a “micronized” material, the water-insoluble drug compound can be sized in the presence of lecithin and optionally a surfactant. If the water-insoluble drug compound is sized prior to mixing with lecithin, then the mixing with lecithin step must occur prior to combining the water-insoluble drug compound with the remainder of the composition. Particle sizing techniques are known in the art and include ball milling, homogenization and micronization. As used herein, “mixing” includes simple mixing as well as sizing procedures.

The lecithin ingredient should be dispersed in water at a temperature above the phase transition temperature for the chosen grade of lecithin. In the case of phospholipon 90H, the phase transition temperature is approximately 51° C. Therefore, Phospholipon 90H is preferably dispersed at a temperature of approximately 65-70° C. A surfactant, if present, can be dispersed simultaneously with lecithin or added before or after lecithin is fully dispersed. After the surfactant and lecithin are dispersed, the water-insoluble drug compound (preferably micronized) is then dispersed to form a water-insoluble drug compound slurry. The water-insoluble drug compound is preferably added after removing the lecithin dispersion from heat, but before the lecithin dispersion cools to room temperature. The water-insoluble drug compound should be mixed with the lecithin dispersion for approximately 6 to 18 hours or more, preferably 12 hours, before being added to the remainder of the composition.

In a separate vessel, the remainder of excipients are dissolved in water to form an Excipient Solution. Although it is possible to add all of remainder of excipients simultaneously, provided that the vessel contains a sufficient amount of water, sequentially mixing and dispersing/dissolving, with each ingredient being dispersed or dissolved prior to the addition of the next, is preferred. For example, a buffering agent is added to purified water, then a preservative, and finally a tonicity-adjusting agent.

After the Excipient Solution has been prepared, it is combined with the water-insoluble drug compound slurry, then the pH is adjusted with an NaOH or HCI and the batch volume is adjusted with purified water.

The compositions described above are preferably prepared as follows.

1. Add approx. 5-50% of the total batch volume of purified water to a compounding vessel and heat to a temperature above the transition temperature of the chosen grade of lecithin (in the case of Phospholipon 90H the preferred temperature is approximately 65-70° C.).

2. Using a magnetic stir bar, disperse 50% of the total required amount of lecithin (preferably, Phospholipon 90H) and 50% of the total required amount of surfactant (preferably polysorbate 20) into the heated water of Step 1 until uniformly dispersed (generally about 10-20 min.). Remove from heat.

3. Add the water-insoluble drug compound (preferably micronized) before the dispersion of Step 2 cools to room temperature and mix for approximately 12 hrs. (i.e., overnight).

4. Prepare a solution by adding the following components in order and mix well allowing each to disperse or dissolve before adding the next: the remaining 50% of the total amount of lecithin (at elevated temperature), the remaining 50% of the total amount of surfactant, the preservative, the buffer (e.g., glacial acetic acid then sodium acetate (trihydrate)), and the tonicity-adjusting agent.

5. Add the water-insoluble drug dispersion of Step 3 to the solution of Step 4 (while mixing).

6. QS to 90% with purified water.

7. Measure and adjust pH to target pH with 1N NaOH and/or 1N HCl, then QS to 100% with purified water.

The following examples are presented to illustrate further various aspects of the present invention, but are not intended to limit the scope of the invention in any respect.

### EXAMPLES

The formulations shown in Tables 1 and 2 were prepared (ingredient amounts shown as % w/w).

The physical stability of suspension formulations is commonly measured in two ways: resuspendability is assessed by measuring the number of inversions (also called strokes) required to redisperse sedimentation which forms after a sample stands undisturbed for a period of time; and rate of settling is assessed by observing the height in millimeters of the column of sedimentation visible in a sample contained in a cylinder after shaking and then standing for a period of time. In order to record the rate of settling results, the following codes are used (in order of increasing turbidity): C: Clear Supernatant Phase, LM: Light Milky Phase (less dense than Homogeneous phase), H: Homogenous Phase (initial homogeneous phase), D: Dense Phase (more dense than Homogeneous Phase), S: Sediment. Larger sedimentation heights indicate less separation with less supernatant liquid and less compaction of sedimentation. The physical stability of Formulations 1-10 was evaluated according to the methods described above and the results are shown in Tables 3 and 4.

TABLE 1

| | FORMULATION # | | | | |
|---|---|---|---|---|---|
| Ingredient | 1 | 2 | 3 | 4 | 5 |
| Dexamethasone (micronized) | 0.1 | 0.1 | 0.1 | 0.1 | 0.1 |
| Hydroxyethyl Cellulose | — | — | — | 0.3 | 0.05 |
| (NATROSOL 250HR) | | | | | |
| Benzyl Alcohol | 0.9 | 0.9 | 0.9 | 0.9 | 0.9 |
| Sodium Chloride | 0.9 | 0.9 | 0.9 | 0.9 | 0.9 |
| Sodium Acetate (trihydrate) | 0.68 | 0.68 | 0.68 | 0.68 | 0.68 |
| Glacial Acetic Acid | 0.255 | 0.255 | 0.255 | 0.255 | 0.255 |
| Lecithin (Phospholipon 90H) | 0.15 | 0.15 | — | — | — |
| Polysorbate 20 (TWEEN 20) | 0.1 | — | 0.1 | 0.1 | 0.1 |
| Sodium Hydroxide | QS to pH 4.7 | QS to pH 4.7 | QS to pH 4.7 | QS to pH 4.7 | QS to pH 4.7 |
| Hydrochloric Acid | | | | | |
| Purified water | QS to 100 | QS to 100 | QS to 100 | QS to 100 | QS to 100 |

TABLE 2

| | Formulation # | | | | |
|---|---|---|---|---|---|
| Ingredient | 6 | 7 | 8 | 9 | 10 |
| Dexamethasone Beloxil | 0.1 | 0.1 | 0.1 | 0.1 | 0.1 |
| Hydroxyethyl Cellulose | — | — | — | 0.3 | 0.05 |
| (NATROSOL 250HR) | | | | | |
| Benzyl Alcohol | 0.9 | 0.9 | 0.9 | 0.9 | 0.9 |
| Sodium Chloride | 0.9 | 0.9 | 0.9 | 0.9 | 0.9 |
| Sodium Acetate (trihydrate) | 0.68 | 0.68 | 0.68 | 0.68 | 0.68 |
| Glacial Acetic Acid | 0.255 | 0.255 | 0.255 | 0.255 | 0.255 |
| Lecithin (Phospholipon 90H) | 0.15 | 0.15 | — | — | — |
| Polysorbate 20 (TWEEN 20) | 0.1 | — | 0.1 | 0.1 | 0.1 |
| Sodium Hydroxide | pH Adjust to 4.7 | pH Adjust to 4.7 | pH Adjust to 4.7 | pH Adjust to 4.7 | pH Adjust to 4.7 |
| Hydrochloric Acid | | | | | |
| Purified water | QS to 100 | QS to 100 | QS to 100 | QS to 100 | QS to 100 |

TABLE 3
Resuspendability

| Resuspendability | 1 | 2 | 3 | 4 | 5 | 6 | 7 | 8 | 9 | 10 |
|---|---|---|---|---|---|---|---|---|---|---|
| Real Time | | | | | | | | | | |
| # Inversions after 4 | 1 | 2 | 1 | 60 | 3 | 1 | 2 | 5 | 35 | 3 |
| days standing | | | | | | | | | | |
| Accelerated | | | | | | | | | | |
| 30 min. @ 500 rpm | 2, 2 | 2, 3 | 1, 1 | 28, 29 | 2, 3 | 2, 2 | 4, 4 | 3, 4 | 33, 30 | 3, 2 |
| # Inversions | | | | | | | | | | |
| Wrist shaking (sec.) | <1, <1 | <1, <1 | <1, <1 | 3, 4 | <1, <1 | <1, <1 | <1, <1 | <1, <1 | 2, 2 | <1, <1 |

TABLE 4
Rate of Settling

| | FORMULATION # | | | | |
|---|---|---|---|---|---|
| Time | 1 | 2 | 3 | 4 | 5 |
| Initial | 0-10 ml:H | 0-10 ml:H | 0-10 ml:H | 0-10 ml:H | 0-10 ml:H |
| 5 min | 0-9.5 ml:LM | 0-9.5 ml:LM | 0-0.2 ml:S | 0-10 ml:H | 0-0.2 ml:S |
| | 9.5-10 ml:C | 9.5-10 ml:C | 0.2-8.5 ml:LM | (no sediment) | 0.2-9.8 ml:LM |
| | (no sediment) | (flocculated susp, | 8.5-10 ml:C | | 9.8-10 ml:C |
| | | no sediment) | | | |
| 10 min | 0-9.5 ml:LM | 0-9.5 ml:LM | 0-0.2 ml:S | 0-10 ml:H | 0-0.2 ml:S |
| | 9.5-10 ml:C | 9.5-10 ml:C | 0.2-8 ml:LM | (no sediment) | 0.2-9.8 ml:LM |
| | (no sediment) | (flocculated susp, | 8-10 ml:C | | 9.8-10 ml:C |
| | | no sediment) | | | |
| 15 min | 0-0.05 ml:S | 0-8 ml:D | 0-0.3 ml:S | 0-0.01 ml:S | 0-0.2 ml:S |
| | 0.05-9.5 ml:LM | 8-9 ml:LM | 0.3-7.5 ml:LM | 0.01-9.7 ml:LM | 0.2-8.2 ml:LM |
| | 9.5-10 ml:C | 9-10 ml:C | (very few particles) | 9.7-10 ml:C | (few particles) |
| | | | 7.5-10 ml:C | | 8.2-10 ml:C |
| 20 min | 0-0.05 ml:S | 0-8 ml:D | 0-0.3 ml:S | 0-0.01 ml:S | 0-0.2 ml:S |
| | 0.05-9.5 ml:LM | 8-9 ml:LM | 0.3-7 ml:LM | 0.01-9.7 ml:LM | 0.2-8.2 ml:LM |
| | 9.5-10 ml:C | 9-10 ml:C | (very few particles) | 9.7-10 ml:C | (few particles) |
| | | | 7-10 ml:C | | 8.2-10 ml:C |
| 30 min | 0-0.1 ml:S | 0-3 ml:S | 0-0.3 ml:S | 0-0.01 ml:S | 0-0.2 ml:S |
| | 0.1-9.5 ml:LM | (flocculated sediment) | 0.3-4 ml:LM | 0.01-9.7 ml:LM | 0.2-8.2 ml:LM |
| | 9.5-10 ml:C | 3-9 ml:LM | (very few particles) | 9.7-10 ml:C | (few particles) |
| | | 9-10 ml:C | 4-10 ml:C | | 8.2-10 ml:C |
| 45 min | 0-0.1 ml:S | 0-2.3 ml:S | 0-0.3 ml:S | 0-0.01 ml:S | 0-0.2 ml:S |
| | 0.1-9.5 ml:LM | 2.3-9 ml:LM | 0.3-4 ml:LM | 0.01-9.7 ml:LM | 0.2-8.2 ml:LM |
| | 9.5-10 ml:C | (very few particles) | (very few particles) | 9.7-10 ml:C | (very few particles) |
| | | 9-10 ml:C | 4-10 ml:C | | 8.2-10 ml:C |
| | | (no particles) | | | |
| 1 hr | 0-0.1 ml:S | 0-2 ml:S | 0-0.3 ml:S | 0-0.01 ml:S | 0-0.2 ml:S |
| | 0.1-9.5 ml:LM | 2-9 ml:LM | 0.3-10 ml:C | 0.01 -9.7 ml:LM | 0.2-8.2 ml:LM |
| | (flocculated) | (very few particles) | | 9.7-10 ml:C | (very few particles) |
| | 9.5-10 ml:C | 9-10 ml:C | | | 8.2-10 ml:C |
| 2 hrs | 0-0.1 ml:S | 0-1.5 ml:S | 0-0.3 ml:S | 0-0.01 ml:S | 0-0.2 ml:S |
| | 0.1-9.5 ml:LM | 1.5-10 ml:C | 0.3-10 ml:C | 0.01-9.5 ml:LM | 0.2-10 ml:C |
| | (flocculated) | | | 9.5-10 ml:C | |
| | 9.5-10 ml:C | | | | |
| 3 hrs | 0-0.3 ml:S | 0-1.2 ml:S | 0-0.3 ml:S | 0-0.01 ml:S | 0-0.2 ml:S |
| | 0.3-9 ml:LM | 1.2-10 ml:C | 0.3-10 ml:C | 0.01-9.5 ml:LM | 0.2-10 ml:C |
| | (flocculated) | | | 9.5-10 ml:C | |
| | 9-10 ml:C | | | | |
| 1 Day | 0-3.8 ml:S | 0-1 ml:S | 0-0.2 ml:S | 0-0.1 ml:S | 0-0.2 ml:S |
| | 3.8-10 ml:C | 1-10 ml:C | 0.2-10 ml:C | 0.1-10 ml:C | 0.2-10 ml:C |
| | | | | (with some | |
| | | | | haziness present) | |
| 3 Days | 0-2.2 ml:S | 0-1 ml:S | 0-0.2 ml:S | 0-0.1 ml:S | 0-0.2 ml:S |
| | 2.2-10 ml:C | 1-10 ml:C | 0.2-10 ml:C | 0.1-10 ml:C | 0.2-10 ml:C |
| | FORMULATION # | | | | |
| Time | 6 | 7 | 8 | 9 | 10 |
| Initial | 0-10 ml:H | 0-10 ml:H | 0-10 ml:H | 0-10 ml:H | 0-10 ml:H |
| 5 min | 0-10 ml:H | 0-10 ml:H | 0-10 ml:H | 0-10 ml:H | 0-10 ml:H |
| | (No Sediment) | (No Sediment) | (No Sediment) | (No Sediment) | (No Sediment) |
| 10 min | 0-10 ml:H | 0-10 ml:D | 0-10 ml:H | 0-10 ml:H | 0-10 ml:H |
| | (No Sediment) | (Flocculated Suspension) | (No Sediment) | (No Sediment) | (No Sediment) |
| | | (No Sediment) | | | |
| 15 min | 0-10 ml:H | 0-1 ml:S | 0-9.6 ml:LM | 0-10 ml:H | 0-10 ml:LM |
| | (No Sediment) | (Flocculated Sediment) | 9.6-10 ml:C | (No Sediment) | (light sediment on bottom) |
| | | 1-10 ml:D | (light sediment on bottom) | | |
| 20 min | 0-10 ml:H | 0-1 ml:S | 0-0.05 ml:S | 0-10 ml:H | 0-0.05 ml:S |
| | (No Sediment) | (Flocculated Sediment) | 0.05-9.5 ml:LM | (No Sediment) | 0.05-9.8 ml:LM |
| | | 1-9 ml:D | 9.5-10 ml:C | | 9.8-10 ml:C |
| | | 9-10 ml:LM | | | |
| 30 min | 0-10 ml:H | 0-1.9 ml:S | 0-0.05 ml:S | 0-10 ml:H | 0-0.08 ml:S |
| | (No Sediment) | (Flocculated Sediment) | 0.05-9.5 ml:LM | (No Sediment) | 0.08-9.7 ml:LM |
| | | 1.9-7.0 ml:D | 9.5-10 ml:C | | 9.7-10 ml:C |
| | | 7-10 ml:LM | | | |
| 45 min | 0-10 ml:H | 0-1.9:S | 0-0.05 ml:S | 0-10 ml:H | 0-0.08 ml:S |
| | (No Sediment) | (Flocculated Sediment) | 0.05-9.5 ml:LM | (No Sediment) | 0.08-9.7 ml:LM |
| | | 1.9-10 ml:C | 9.5-10 ml:C | | 9.7-10 ml:C |
| 1 Hr | 0-10 ml:H | 0-1.7 ml:S | 0-0.05 ml:S | 0-10 ml:H | 0-0.08 ml:S |
| | (No Sediment) | (Flocculated Sediment) | 0.05-9.5 ml:LM | (No Sediment) | 0.08-9.7 ml:LM |
| | | 1.7-10 ml:C | 9.5-10 ml:C | | 9.7-10 ml:C |
| 2 Hrs | 0-9.7 ml:H | 0-1.3 ml:S | 0-0.05 ml:S | 0-9.7 ml:H | 0-0.08 ml:S |
| | 9.7-10 ml:C | (Flocculated Suspension | 0.05-9.5 ml:LM | 9.7-10 ml:C | 0.08-10 ml:C |
| | (Flocculated Suspension) | 1.3-10 ml:C | 9.5-10 ml:C | | |
| 3 Hrs | 0-9 ml:H | 0-1 ml:S | 0-0.1 ml:S | 0-9.7 ml:H | 0-0.1 ml:S |
| | 9-10 ml:C | (Flocculated Sediment) | 0.1-10 ml:C | 9.7-10 ml:C | 0.1-10 ml:C |
| | (Flocculated Suspension) | 1-10 ml:C | | | |
| 1 Day | 0-3.3 ml S | 0-0.8 ml:S | 0-0.1 ml:S | 0-0.05 ml:S | 0-0.1 ml:S |
| | (Flocculated Sediment) | (Flocculated Sediment) | 0.1-10 ml:C | 0.05-10 ml:C | 0.1-10 ml:C |
| | 3.3-10 ml:C | 0.8-10 ml:C | | | |
| 3 Days | 0-2.1 ml:S | 0-0.7 ml:S | 0-0.1 ml:S | 0-0.1 ml:S | 0-0.1 ml:S |
| | (Flocculated Sediment) | (Flocculated Sediment) | 0.1-10 ml:C | 0.1-10 ml:C | 0.1-10 ml:C |
| | 2.1-10 ml:C | 0.7-10 ml:C | | | |

The results shown in Tables 3 and 4 demonstrate that the compositions of the present invention (Formulation #'s 1, 2, 6 and 7) have equivalent or superior physical stability to compositions containing a conventional polymeric suspending agent (Formulation #'s 4, 5, 9 and 10). When compared to Formulation #'s 5 and 10 (containing a relatively low concentration of a polymeric suspending agent such that after settling, the formulations would be relatively easy to resuspend), the formulations of the present invention have approximately equivalent resuspendability results but superior rate of settling results. See, for example, the data shown after 2 hours of settling. When compared to Formulation #'s 4 and 9 (containing a relatively high concentration of a polymeric suspending agent such that the rate of settling would be relatively low), the formulations of the present invention have approximately equivalent or superior rate of settling results but superior resuspendability results (2-4 inversions for Formulation #'s 1, 2, 6 and 7, but 28-33 inversions for Formulation #'s 4 and 9). See, for example, the data shown after 1 day of settling (where the greater the height of the “Sediment” phase, the more flocculated and easier to resuspend the formulation). Comparing the formulations of the present invention to Formulation #'s 3 and 8 (containing a surfactant but no lecithin or polymeric suspending agent), the resuspendability results were approximately equivalent, but the rate of settling results of the formulations of the present invention were superior. See, for example, the data shown after 1 day of settling.

The invention has been described by reference to certain preferred embodiments; however, it should be understood that it may be embodied in other specific forms or variations thereof without departing from its spirit or essential characteristics. The embodiments described above are therefore considered to be illustrative in all respects and not restrictive, the scope of the invention being indicated by the appended claims rather than by the foregoing description.

We claim:

1. An aqueous pharmaceutical suspension composition comprising one or more water-insoluble drug compounds and a physical-stability enhancing additive consisting essentially of lecithin.

2. The composition of claim 1 wherein the water-insoluble drug compound is present in an amount from about 0.001-5%.

3. The composition of claim 1 wherein the water-insoluble drug compound is a steroid.

4. The composition of claim 3 wherein the steroid is selected from the group consisting of dexamethasone; rimexolone; prednisolone; hydrocortisone; fluticasone propionate; budesonide; mometasone furoate monohydrate; and dexamethasone beloxil.

5. The composition of claim 1 wherein the water-insoluble drug compound is selected from the group consisting of griseofulvin; carbamazepin; clofibrate; ketoprofen; 5-flurouracil; flurbiprofen; mefanamic acid; flufenamic acid; and crystalline beta escinic acid.

6. The composition of claim 1 wherein the lecithin is present in an amount from about 0.01-5%.

7. The composition of claim 6 wherein the lecithin is present in an amount from about 0.01-2%.

8. The composition of claim 1 wherein the lecithin is selected from the group consisting of phosphatidylcholine; phosphatidylglycerol; phosphatidylinositol; sphingomyelin; phosphatidylethanolamine; distearoylphosphatidyl choline; dipalmitoylphosphatidyl choline; and dimirystoylphosphatidyl choline.

9. The composition of claim 1 further comprising a surfactant.

10. The composition of claim 9 wherein the surfactant is selected from the group consisting of polysorbate 20-80 surfactants.

11. The composition of claim 10 wherein the surfactant is present in an amount from about 0.01-2%.

12. The composition of claim 9 further comprising one or more excipients selected from the group consisting of tonicity-adjusting agents; preservatives; buffering agents; chelating agents; anti-oxidants.

13. A method of preparing an aqueous pharmaceutical suspension composition comprising one or more water-insoluble drug compounds and a physical-stability enhancing additive consisting essentially of lecithin wherein the one or more water-insoluble drug compounds are mixed with lecithin and optionally a surfactant to form a water-insoluble drug compound slurry prior to being combined with any other excipients.

14. The method of claim 13 wherein the one or more water-insoluble drug compounds are mixed with lecithin and a surfactant for about 6 to 18 hours prior to being combined with any other excipients.

15. The composition of claim 10 wherein the water-insoluble drug compound is a steroid and is present in an amount from about 0.001-5%.

16. The method of claim 13 wherein the lecithin is present in an amount from about 0.01-5%.

17. The method of claim 16 wherein the lecithin is selected from the group consisting of phosphatidylcholine; phosphatidylglycerol; phosphatidylinositol; sphingomyelin; phosphatidylethanolamine; distearoylphosphatidyl choline; dipalmitoylphosphatidyl choline; and dimirystoylphosphatidyl choline.

18. The method of claim 13 wherein the surfactant is selected from the group consisting of polysorbate 20-80 surfactants.

19. The method of claim 18 wherein the surfactant is present in an amount from about 0.01-2%.

20. The method of claim 13 wherein the aqueous pharmaceutical suspension composition comprises one or more excipients selected from the group consisting of tonicity-adjusting agents; preservatives; buffering agents; chelating agents; anti-oxidants.
